(19) United States
(12) Patent Application Publication
(10) Pub. No.: US 2006/0167104 A1
(43) Pub. Date: Jul. 27, 2006

(54) Method for extracting a novel compound from the root of a plant of the pentadiplandraceae family and use of N,N'-dibenzylthiourea as a medicine

(76) Inventor: Thomas Wandji, Yaounde (CM)

Correspondence Address: LERNER, DAVID, LITTENBERG,;KRUMHOLZ & MENTLIK, 600 SOUTH AVENUE WEST, WESTFIELD, NJ, 07090, US

(21) Appl. No.: 10/959,458

(22) Filed: Oct. 5, 2004

Related U.S. Application Data
(63) Continuation-in-part of application No. PCT/OA03/00001, filed on Mar. 27, 2003.

(30) Foreign Application Priority Data
Apr. 5, 2002 (OA) 1200200098

(51) Int. Cl. A61K 31/17

(52) U.S. Cl. 514/585; 564/26

(57) ABSTRACT

The invention concerns a method for extracting a biological antioxidant form a plant of the pentadiplandraceae family genus Pentadiplandra brazzeana, brazzeana variety. The analysis of said biological oxidant has shown that said molecule is N,N′-dibenzylthiourea. The extraction of synthesis of said molecule should be carried out without any contact with oxygen to avoid destruction of said active sites by oxygen. The preservation in hydroalcoholic solution of N,N′-dibenzylthiourea when it is extracted from Pentadiplandra brazzeana, a brazzeana variety or its synthesis in laboratory enables preservation of the active sites of said substance, of (—NH_) groups from which those pharmacological properties are derived.

The present invention is related to the use of the compound N,N′-dibenzylthiourea extracted from Pentadiplandra Brazzeana, variety Brazzeana, in the therapeutic treatment of man and animals and to an extraction procedure of said compound.

This plant is a liana found in forests of Central Africa. It is well known to botanists and ethnobotanists, its roots are used as a condiment by some Central African populations.

Phytochemical studies have been made on this plant, but none have isolated N,N′-dibenzylthiourea and demonstrated its biological antioxidant potential and shown its mode of action.

Extraction

The extraction procedure linked to the present invention allows N,N′-dibenzylthiourea to be isolated. 1 kg of ground and dried roots are placed in a yellow coloured 2-litre flask. 1 litre of ethyl alcohol at 95° is added as a solvent.

The stoppered flask is placed on a mechanical shaker and the flask is shaken for 4 hours to fully extract the compound. The shaker is stopped and the flask is left standing for 1 hour. The liquid is filtered and the lemon-yellow coloured filtrate is recovered. The alcohol is removed by vacuum distillation at about 15 mm Hg pressure in a rotary evaporator with water bath thermostatically regulated at 40°. The residue should not be allowed to dry.

To obtain the N,N′-dibenzylthiourea, the concentrated alcoholic solution is placed in a decanter into which is poured 250 ml of carbon tetrachloride and the whole is shaken by hand for one hour. After standing, two phases appear in the decanter.

The hydroalcoholic phase contains N,N′-dibenzylthiourea, which is not soluble in the carbon tetrachloride.

We have observed that a few ml of this solution evaporated to dryness gives a blackish, sticky residue in the presence of oxygen from the air. The structural study of this residue shows that it is a N,N′-dibenzylthiourea polymer without pharmacological effect.

The mechanism of polymer formation:

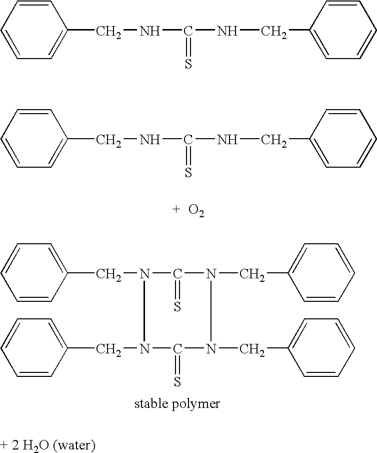

The outcome of this reaction is the formation of a polymer from 2 molecules of N,N′-dibenzylthiourea with the loss of 4 hydrogen atoms which produce 2 molecules of water on contact with the air.

We have thus demonstrated after structural analysis the presence of a biological antioxidant in the root of the Pentadiplandra Brazzeana variety Brazzeana which is N,N′-dibenzylthiourea in solution.

So that this substance is used as a medicine for humans or animals, we have demonstrated the in vitro action of this biological anti-oxidant on the hydroxyl radical (OH°) which by its aggressivity alters the state and functioning of numerous cells of human and animal organisms.

We used the Fentom method. This method is based on the degradation of deoxyribose by free radicals generated by the xanthine/xanthine oxidase system. This degradation leads to the formation of malondialdehyde which is measured by colorimetry.

In this technique, the trapping of free hydroxyl radicals is translated into a decrease in the formation of malondialdehyde.

N,N′-dibenzylthiourea in solutions of concentration 20, 10 and 5% decreases the production of malondialdehyde by 65, 59 and 51% respectively in relation to the control containing no free hydroxyl radical trapper.

The extraction procedure linked to the present invention allows N,N′-dibenzylthiourea to be isolated. 1 kg of ground and dried roots are placed in a yellow coloured 2-litre flask. 1 litre of ethyl alcohol at 95° is added as a solvent.

The stoppered flask is placed on a mechanical shaker and the flask is shaken for 4 hours to fully extract the compound. The shaker is stopped and the flask is left standing for 1 hour. The liquid is filtered and the lemon-yellow coloured filtrate is recovered. The alcohol is removed by vacuum distillation at about 15 mm Hg pressure in a rotary evaporator with water bath thermostatically regulated at 40°. The residue should not be allowed to dry.

Therapeutic Indications

This medicine is indicated for the treatment of all diseases caused by the presence of the oxygenated free radical (hydroxyl radical: .OH) in the human or animal organism, notably Alzheimer's disease, Parkinson's disease, cancers affecting the elderly and children, severe avitaminoses affecting adults and children (kwashiorkor), cardiovascular disease, diabetes and complications, aging, diseases caused by smoking.

The hydroxyl radical .OH is responsible for the alteration of the state and functioning of numerous cells.

It causes the degeneration of cells independent of their origin. Until now the medicines used in the treatment of these diseases were meant to treat only their symptom.

This is how dopaminergics (apomorphine), tranquilisers, sleeping pills, antidepressants, neuroleptics, oxygenators and vasodilators are used.

Treatment by N,N′-dibenzylthiourea is destined to rid the organism of the hydroxyl radical (OH). This preventative and curative treatment regenerates dead tissues and re-establishes normal functioning of the cells.

Free radicals are atoms, groups of atoms or molecules possessing an unpaired or singlet electron on an external orbit. The pairing tendency of these external orbital electrons explains their biological aggressivity.

By removing this electron from another molecule, immediately transformed in its turn into a free radical, they provoke chain reactions resulting in the alteration of numerous cellular structures.

The appearance of the hydroxyl radical in the organism happens as follows: hydrogen peroxide appears naturally in the organism but is regularly destroyed by catalase in such a way that the formation reaction of this hydrogen peroxide from water and oxygen is reversible.
H₂O₂⇄H₂O+½O₂ (by catalase action)

In children from 0 to 5 years of age and the elderly, insufficient secretion of catalase is observed and the above reaction is no longer reversible—hydrogen peroxide undergoes the Fenton reaction during which decomposes into a harmless OH⁻ ion and a very reactive hydroxyl radical OH.

This reaction occurs in the presence of Fe²⁺ present in the human or animal organism
Fe²⁺+H₂O₂→Fe³⁺+OH⁻+.OH

The appearance of the OH radical in the organism may also be induced directly by heat, light or energetic radiation. The alteration of numerous cellular structures generally affects nuclei: modification of structures and noble elements particularly ribonucleic and deoxyribonucleic acids as well as the double bonds of polyunsaturated fatty acids in cell membranes.

The diseases in humans and animals which constitute a therapeutic indication for N,N′-dibenzylthiourea are caused in the organism by the aggressivity of the hydroxyl radical.

The struggle against these diseases consists of using biological molecules able to give a hydrogen atom to the hydroxyl radical, to transform it into water. For example: N,N′-dibenzylthiourea gives mobile H or mobile Hydrogen to the hydroxyl radical according to the following mechanism:

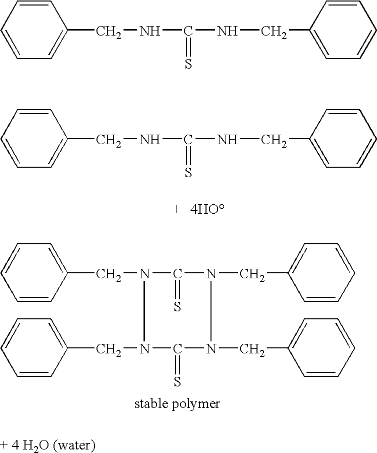

The medicine, the object of the present invention having as its active principle N,N′-dibenzylthiourea extracted from Pentadiplandra Brazzeana variety Brazzeana supplies the “H” to the hydroxyl radical .OH thus transforming it into water, which is eliminated from the organism in the urine, and the polymer formed which is neutral is also eliminated in the urine.

The advantage of this antioxidant is that it is biological.

N,N′-dibenzylthiourea is non-toxic apart from its ocytocic action on the gravid uterus.

This medicine is presented under the following galenic forms:

Syrup
COMPOSITION

| | | |
|---|---|---|
| N,N′-dibenzylthiourea: | 108 | mg |
| Excipient: sucrose syrup | SQ 250 | ml |

DOSAGE

| | | |
|---|---|---|
| Adult: | 2 table spoons | Morning and evening |
| Child: | 2 tea spoons | Morning and evening |

Other orally (capsules, pills) and rectally (suppositories) administered forms may be manufactured. It is simply necessary to protect the N,N′-dibenzylthiourea from the air.

1-11. (canceled)

12. A method of extracting N,N′-dibenzylthiourea from the root of a plant of the family of Pentadiplandra Brazzeana, variety Brazzeana, comprising the steps of:
grinding the roots;
drying the ground roots;
adding ethyl alcohol at 95°;
shaking the mixture;
filtering the mixture and collecting the filtrate;
distilling the filtrate under vacuum at a temperature of about 40° C. until a concentrated alcoholic solution is obtained, while avoiding having a dry residue;
adding carbon tetrachloride to the concentrated alcoholic solution; and
decanting and recovering the concentrated alcoholic solution containing said N,N′-dibenzylthiourea.

13. N,N′-dibenzylthiourea produced according to the process of claim 12.

14. A hydroalcoholic solution comprising the N,N′-dibenzylthiourea of claim 13.

15. A method of preparing an antioxidant drug comprising the step of using the N,N′-dibenzylthiourea of claim 13.

16. The method of preparing the antioxidant drug of claim 15 comprising dissolving said N,N′-dibenzylthiourea in a hydroalcoholic solution.

17. An antioxidant comprising the N,N′-dibenzylthiourea of claim 13.

18. An antioxidant comprising the hydroalcoholic solution of claim 14.

19. A method of treating a patient having a pathology caused by the alteration of the state and functioning of animal or human organism cells by the action of hydroxyl radical comprising administering to said patient an effective amount of the N,N′-dibenzylthiourea of claim 13.

20. A method of treating a patient having a pathology selected from the group consisting of Alzheimer's disease, Parkinson's disease, cancers affecting the elderly and children, severe avitaminoses affecting adults and children (kwashiorkor), cardiovascular disease, diabetes and its complications, aging, and diseases caused by smoking, comprising administering to said patient an effective amount of the N,N′dibenzylthiourea of claim 13.

21. A pharmaceutical composition for use as a medicament comprising N,N′-dibenzylthiourea and sucrose syrup.

22. The pharmaceutical composition of claim 21 wherein said N,N′-dibenzylthiourea is present in an amount of 108 mg said sucrose syrup is present in the amount of 250 mg.

23. The pharmaceutical composition of claim 21 in a form for administration selected from the group consisting of syrup, capsule, suppository, and pill.
